(19) United States
(12) Patent Application Publication
(10) Pub. No.: US 2010/0036203 A1
(43) Pub. Date: Feb. 11, 2010

(54) ENDOSCOPE SYSTEM

(75) Inventors: Masaya Nakaoka, Tokyo (JP); Koki Morishita, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/518,798

(22) PCT Filed: Nov. 16, 2007
(86) PCT No.: PCT/JP2007/072261

§ 371 Date: Jun. 11, 2009

(30) Foreign Application Priority Data
Dec. 14, 2006 (JP) 2006-337596

(51) Int. Cl. A61B 1/06

(52) U.S. Cl. 600/178

(57) ABSTRACT

It is possible to acquire a fluorescence distribution image for each fluorescent agent from a fluorescence image acquired in a mixed state, thus improving the diagnostic performance of cancer cells. There is provided an endoscope system (1) configured to insert at least a part into a body cavity of a living body and to acquire an image of an image-acquisition subject in the body cavity, the endoscope system including a light source unit (10) configured to emit excitation light for exciting two or more different types of fluorescent agents having different optical characteristics; two or more image-acquisition units (14a, 14b) provided in a section inserted in the body cavity and configured to simultaneously capture fluorescence emitted from the image-acquisition subject as fluorescence in two or more different wavelength bands; a storage unit configured to store information associated with the relative relationship between the intensity of fluorescence generated when excited by the excitation light and the concentrations of the fluorescent agents; and a concentration-information calculating unit (18) configured to calculate and output concentration information of the fluorescent agents on the basis of fluorescence intensity of images in two or more wavelength bands captured by the image-acquisition units and the information associated with the relative relationship stored in the storage unit.

### TECHNICAL FIELD

The present invention relates to an endoscope system.

### BACKGROUND ART

Conventionally, diagnosis and treatment in which a fluorescent material having affinity to a disease, such as cancer, is injected in advance into the body of a test subject and excitation light that excites the fluorescent material is emitted to detect fluorescence from the fluorescent material accumulated in the diseased site have been attracting attention. According to this diagnosis method, since intense fluorescence is radiated from the diseased site, the presence of a lesion can be determined from the brightness of a fluorescence image.

Patent Document 1 discloses an endoscope apparatus that diagnoses cancer cells using such a method.

Patent Document 1:
Japanese Unexamined Patent Application, Publication No. HEI-10-201707

### DISCLOSURE OF INVENTION

Since molecules that are overexpressed in cancer cells are often overexpressed in inflamed areas/benign tumors etc., it is difficult to improve the diagnostic performance of identifying cancer cells with a single type of fluorescent probe.

Many kinds of molecules that are overexpressed due to cancer cells are known. By making a plurality of different types of molecules associated with the cancer cells emit light using fluorescent dyes having different optical characteristic and carrying out examination, the diagnostic performance can be improved.

The present invention provides an endoscope system configured to insert at least thereof a part into a body cavity of a living body and to acquire an image of an image-acquisition subject in the body cavity, the endoscope system including a light source unit configured to emit excitation light for exciting two or more different types of fluorescent agents having different optical characteristics; two or more image-acquisition units provided at a section inserted in the body cavity and configured to simultaneously capture fluorescence emitted from the image-acquisition subject as fluorescence in two or more different wavelength bands; a storage unit configured to store information associated with the relative relationship between the intensity of fluorescence generated when excited by the excitation light and the concentrations of the fluorescent agents; and a concentration-information calculating unit configured to calculate and output concentration information of the fluorescent agents on the basis of fluorescence intensity of images in two or more wavelength bands captured by the image-acquisition units and the information associated with the relative relationship stored in the storage unit.

In the present invention, the information associated with the relative relationship may be information about the ratio of the intensity of the fluorescence generated when excited by the excitation light and the concentration of the fluorescent agents.

In the present invention, a display configured to display the concentration information calculated and output by the concentration-information calculating unit may be further provided.

In the present invention, the display may have a plurality of channels corresponding to display colors, and the concentration information corresponding to the fluorescent agents may be assigned to and output on the channels.

In the present invention, the wavelength of the excitation light may be set longer than the near-infrared band.

The present invention provides advantages in that the acquisition of a fluorescence distribution image for each fluorescent agent from a fluorescence image acquired in a mixed state is enabled without using a special device, such as a variable spectroscopy device, and in which the diagnostic performance of cancer cells can be improved.

### BRIEF DESCRIPTION OF DRAWINGS

FIG. 1 is a block diagram illustrating the entire configuration of an endoscope system according to a first embodiment of the present invention.

FIG. 2 illustrates the wavelength characteristics of an excitation-light cut filter, a dichroic prism, excitation light, and fluorescence generated by illumination light and excitation light, all used in the endoscope system shown in FIG. 1.

FIG. 3 is a timing chart illustrating the operation of the endoscope system shown in FIG. 1.

FIG. 4 is a timing chart illustrating the operational state of a valve control circuit of the endoscope system shown in FIG. 1.

FIG. 5 illustrates a modification of an image-acquisition unit of the endoscope system shown in FIG. 1.

FIG. 6 illustrates the transmittance characteristics of filters in the image-acquisition unit shown in FIG. 5.

FIG. 7 illustrates another modification of an image-acquisition unit of the endoscope system shown in FIG. 1.

FIG. 8 illustrates the transmittance characteristics of filters in the image-acquisition unit shown in FIG. 7.

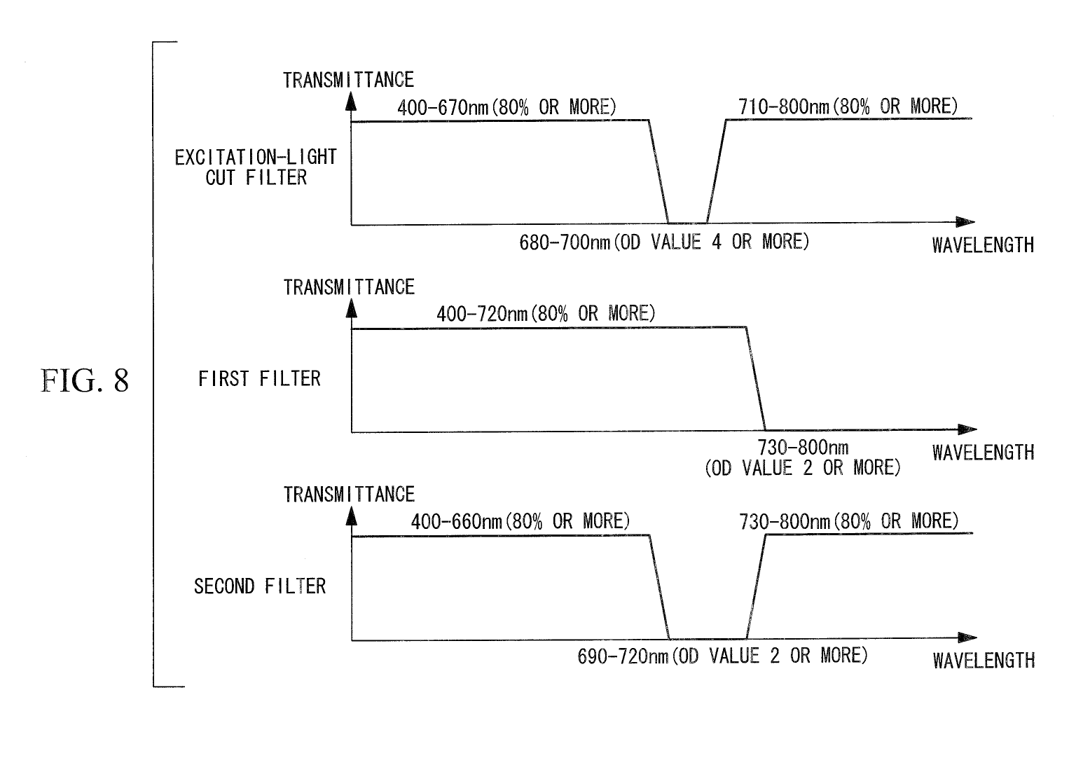

### EXPLANATION OF REFERENCE SIGNS

1: endoscope system
7: display unit (display)
10: excitation light source (light source unit)
14a, 14b: image-acquisition device (image-acquisition unit)
18: image processing circuit (storage unit, concentration-information calculating unit)
N1, N2: concentration information

### BEST MODE FOR CARRYING OUT THE INVENTION

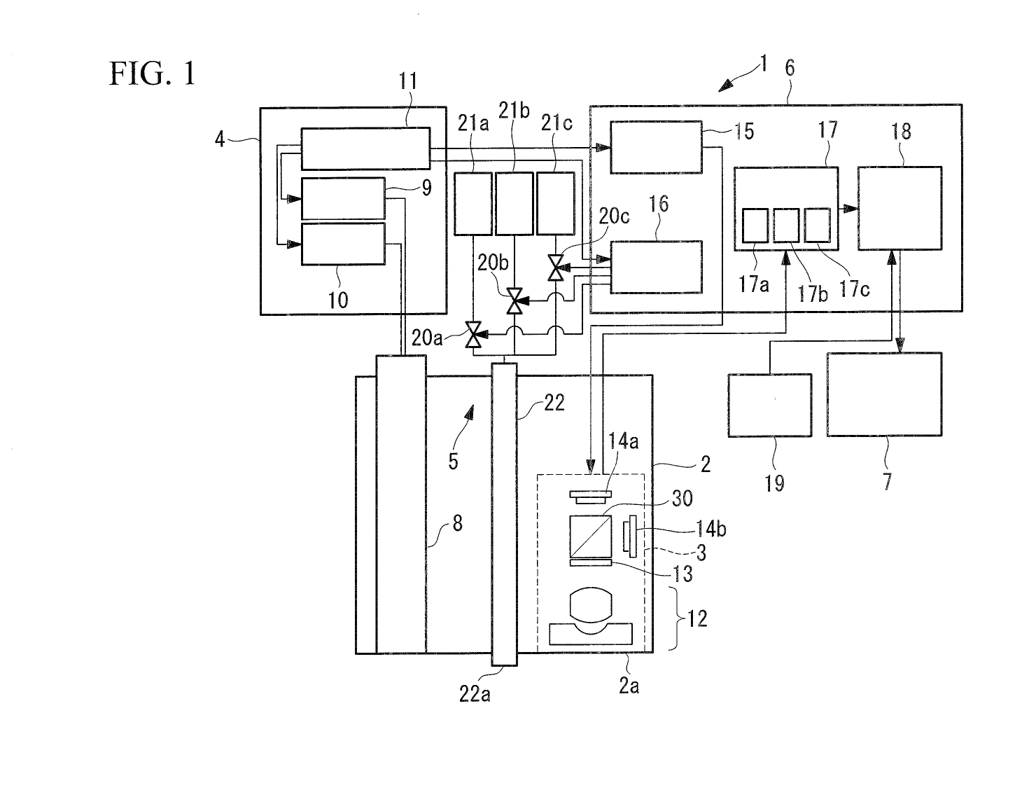

An endoscope system 1 according to a first embodiment of the present invention will be described below with reference to FIGS. 1 to 4.

As shown in FIG. 1, the endoscope system 1 according to this embodiment includes an insertion portion 2 that is inserted into a body cavity of a living body, an image-acquisition unit 3 that is disposed inside the insertion portion 2, a light source unit 4 that generates excitation light and illumination light for normal optical examination, a fluid supplying unit 5 that supplies fluid to be discharged from a tip 2a of the insertion portion 2, a control unit 6 that controls the image-acquisition unit 3, the light source unit 4, and the fluid supplying unit 5, and a display unit (display) 7 that displays an image acquired by the image-acquisition unit 3.

The insertion portion 2 has an extremely thin external shape that allows it to be inserted into the body cavity of a living body and accommodates the image-acquisition unit 3 and a light guide 8 that conveys light from the light source unit 4 to the tip 2a.

The light source unit 4 includes an illumination light source 9 that generates illumination light for illuminating the examined target inside the body cavity and acquiring the reflected light that is reflected at and returned from the examined target, an excitation light source (light source unit) 10 that generates excitation light for generating fluorescence by irradiating the examined target inside the body cavity to excite the fluorescent material present in the examined target, and a light-source control circuit 11 that controls these light sources 9 and 10.

The illumination light source 9, for example, is a combination of a xenon lamp, which is not shown in the drawings, and color filters that can be switched sequentially, and generates red (R), green (G), and blue (B) illumination light in sequence.

The excitation light source 10 is, for example, a semiconductor laser that emits excitation light having a peak wavelength of 690±5 nm. This excitation light can excite an AlexaFluor680 (manufactured by MolecularProbes)-based fluorescent probe. At the same time, the excitation light can also excite an AlexaFluor700 (manufactured by MolecularProbes)-based fluorescent probe.

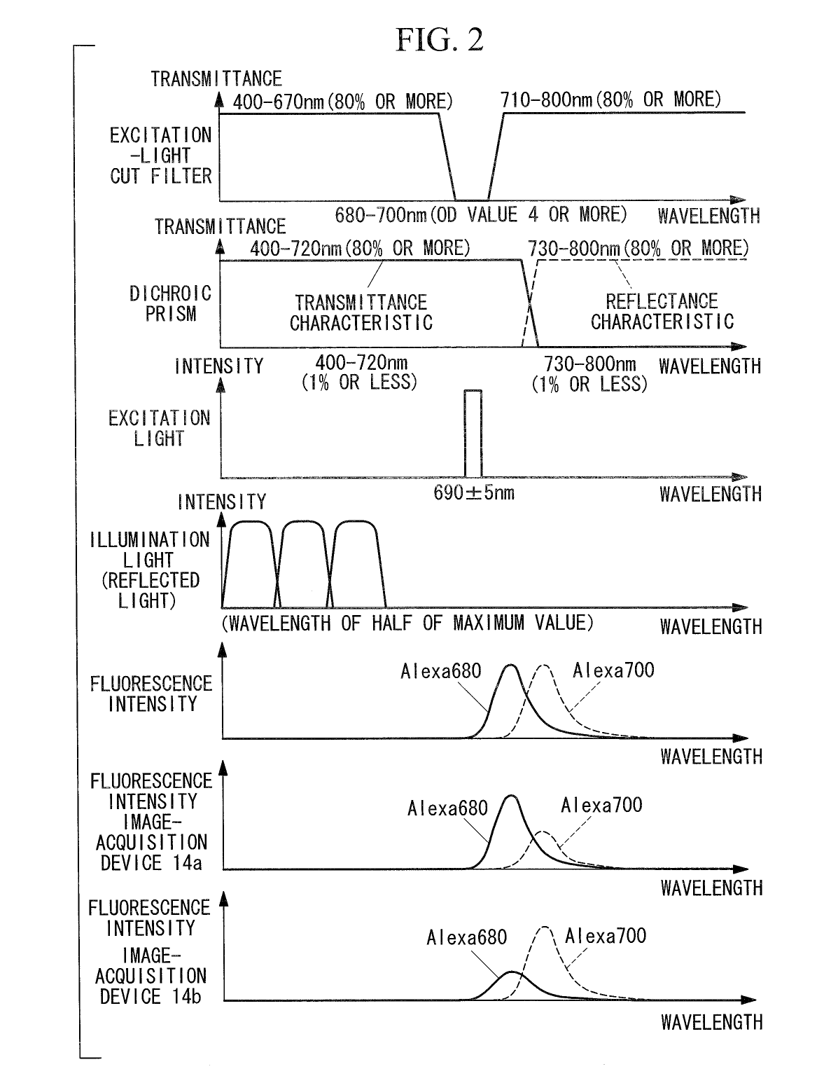

As shown in FIG. 2, the wavelength bands of the fluorescence generated by exciting AlexaFluor680 and AlexaFluor700 overlap. Therefore, when the examined target is irradiated with excitation light while the two fluorescent probes are sprayed onto the examined target, the two fluorescent probes are simultaneously excited, simultaneously generating fluorescence from the two different types of fluorescent probes.

The light-source control circuit 11 alternately turns on and off the illumination light source 9 and the excitation light source 10 at a predetermined timing according to a timing chart described below.

The image-acquisition unit 3 includes an image-acquisition optical system 12 that collects light emitted from the examined target, an excitation-light cut filter 13 that blocks the excitation light emitted from examined target, a dichroic prism 30 that splits the fluorescence from the examined target into two different wavelength bands, and image-acquisition devices (image-acquisition units) 14a and 14b that acquire images of the fluorescence split at the dichroic prism 30 and convert them into electrical signals.

The image-acquisition device 14a receives the fluorescence that is transmitted through the dichroic prism 30, and the image-acquisition device 14b receives the fluorescence reflected at the dichroic prism 30.

As shown in FIG. 2, the excitation-light cut filter 13 has a transmittance characteristic with a transmittance of 80% or more in a wavelength band of 400 nm or more to 670 nm or less, an OD value of 4 or more (=transmittance of 1×10⁻⁴ or less) in a wavelength band of 680 nm or more to 700 nm or less, and a transmittance of 80% or more in a wavelength band of 710 nm or more to 800 nm or less.

Regarding the characteristic of the dichroic prism 30, it has a transmittance of 80% or more and a reflectance of 1% or less in a wavelength band of 400 nm or more to 720 nm or less, and a transmittance of 1% or less and a reflectance of 80% or more in a wavelength band of 730 nm or more to 800 nm or less. At this time, among the fluorescence generated at the examined target, the fluorescence received by the image-acquisition device 14a is mainly in the wavelength band of 720 nm or less, and the fluorescence received by the image-acquisition device 14b is mainly in the wavelength band of 730 nm or more.

As shown in FIG. 1, the control unit 6 includes an image-acquisition device driving circuit (image-acquisition device control circuit) 15 that drives and controls the image-acquisition devices 14a and 14b, a valve control circuit 16 that is described below, frame memories 17 that store image information acquired by the image-acquisition devices 14a and 14b, and an image processing circuit (storage unit, concentration-information calculating unit) 18 that processes the image information stored in the frame memories 17 and outputs it to the display unit 7.

The image processing circuit 18 is connected to an input device 19.

The image-acquisition device driving circuit 15 and the valve control circuit 16 are connected to the light-source control circuit 11 and drive and control the image-acquisition devices 14a and 14b and valves 20a, 20b, and 20c in synchronization with the switching of the illumination light source 9 and the excitation light source 10 by the light-source control circuit 11.

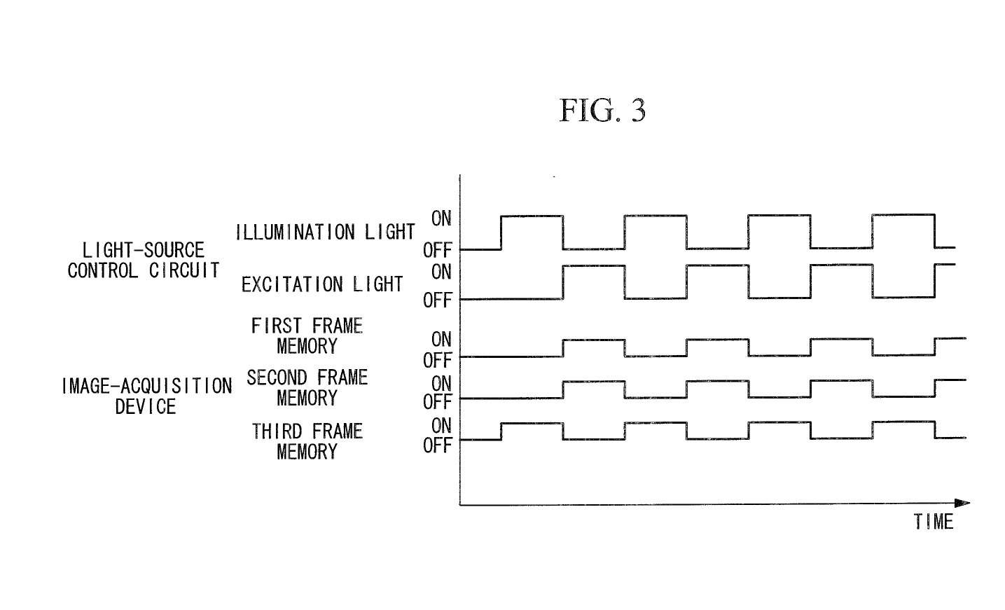

More specifically, as shown in the timing chart in FIG. 3, when excitation light is generated at the excitation light source 10 by operating the light-source control circuit 11, the image-acquisition device driving circuit 15 outputs the image information output from the image-acquisition device 14a to a first frame memory 17a and outputs the image information output from the image-acquisition device 14b to a second frame memory 17b.

When illumination light is generated at the illumination light source 9, the image-acquisition device driving circuit 15 outputs the image information output from the image-acquisition device 14a to a third frame memory 17c.

The image processing circuit 18 receives first fluorescence image information received by the image-acquisition device 14a through excitation light emission and second fluorescence image information received by the image-acquisition device 14b from the first and second frame memories 17a and 17b, respectively, and carries out arithmetic processing. The arithmetic processing at the image processing circuit 18 is carried out as follows.

In other words, the fluorescence intensities per unit concentration acquired from the AlexaFluor680-based fluorescent probe and AlexaFluor700-based fluorescent probe, received by the image-acquisition device 14a when excitation light is emitted, are set as a and b, whereas the fluorescence intensities per unit concentration acquired from the AlexaFluor680-based fluorescent probe and AlexaFluor700-based fluorescent probe, received by the image-acquisition device 14b, are set as c and d.

The relationship represented by Equation 1 holds, where P1 is a fluorescence intensity due to excitation light emission received by the image-acquisition device 14a in a certain region, P2 is a fluorescence intensity received by the image-acquisition device 14b in the same region, and N1 and N2 are concentrations (concentration information) of the AlexaFluor680-based fluorescent probe and the AlexaFluor700-based fluorescent probe, respectively.

$$\begin{matrix}[ Equation 1 ] & \\ ( \begin{matrix}P 1 \\ P 2\end{matrix} ) = ( \begin{matrix}a & b \\ c & d\end{matrix} ) \times ( \begin{matrix}N 1 \\ N 2\end{matrix} ) & ( 1 )\end{matrix}$$

The fluorescence intensities P1 and P2 are measurement results, and by substituting these into Equation 1, the concentrations N1 and N2 of the fluorescent probes can be calculated.

The coefficients a, b, c, and d in Equation 1 can be determined in advance through measurement, etc. and may be input to a processing circuit using the input device 19. Instead, the values determined in advance through measurement, etc. may be stored in a storage device, which is not shown in the drawings, in the control unit during the manufacturing process.

As a result of the calculation, the output concentrations N1 and N2 of the fluorescent probes are output to first (for example, red) and second (for example, green) channels of the display unit 7. The image processing circuit 18 receives reflected-light image information acquired through illumination light emission from the third frame memory 17c and outputs it to the third (for example, blue) channel of the display unit 7.

The fluid supplying unit 5 includes a first tank 21a that retains rinsing water for rinsing the examined target; second and third tanks 21b and 21c that retain first and second fluorescent probe solutions; the valves 20a, 20b, and 20c, which selectively supply and stop the fluid from the tanks 21a, 21b, and 21c; a fluid supplying tube 22 that is connected to the first to third tanks 21a to 21c via the valves 20a to 20c and that supplies the solutions to the tip 2a through the insertion portion 2; and the valve control circuit 16 that is disposed inside the control unit 6 and that controls the valves 20a to 20c. The fluid supplying tube 22 has a tip 22a disposed at the tip 2a of the insertion portion 2 and is capable of spraying the supplied rinsing water or fluorescent probe solutions to the examined target. As the fluid supplying tube 22, a forceps channel provided in the insertion portion 2 may be used.

The valve control circuit 16 is connected to the light-source control circuit 11. The light-source control circuit 11 outputs switching commands for the valves 20a to 20c to the valve control circuit 16 on the basis of the switching timing of the light sources.

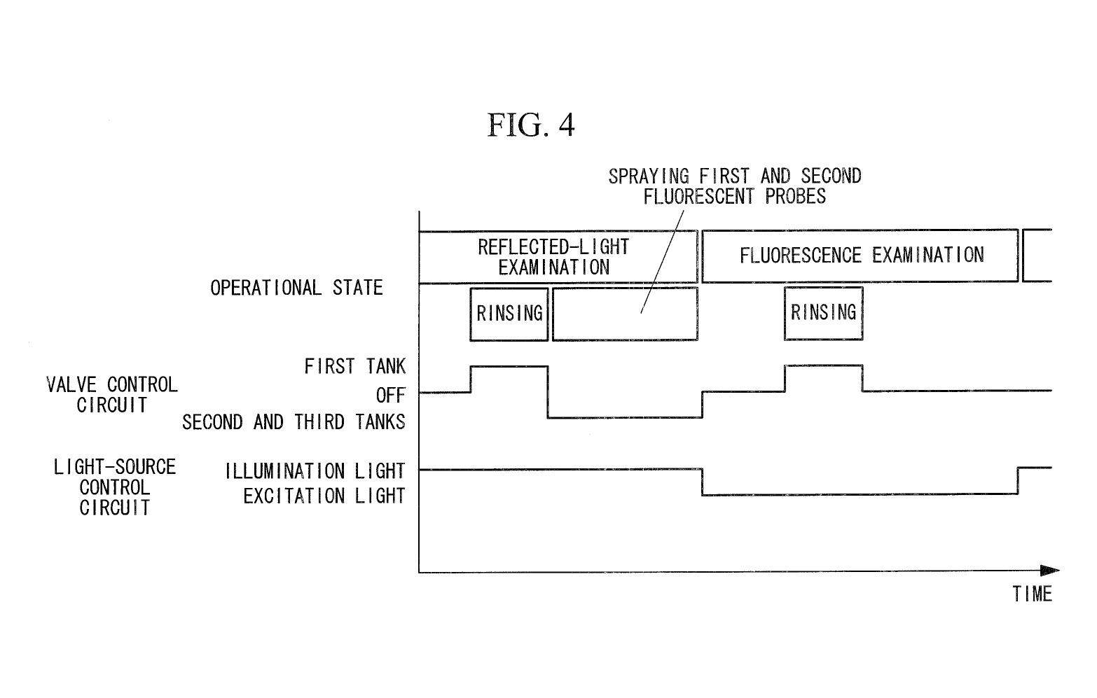

Therefore, as shown in FIG. 4, the valve control circuit 16 controls the valves 20a to 20c so as to open the valve 20a for a predetermined amount of time during reflected-light examination, which is carried out a predetermined amount of time before switching to the excitation light source 10 in response to the switching command from the light-source control circuit 11, in order to discharge the rinsing water retained in the first tank 21a, to close the valve 20a, and to open the valves 20b and 20c in order to spray the fluorescent probe solutions retained in the second and third tanks 21b and 21c.

After spraying the fluorescent probe solutions, the valve control circuit 16 turns off the valves 20a to 20c. Then, after a predetermined amount of time after switching to the excitation light source 10 in response to the switching command from the light-source control circuit 11, the valve control circuit 16 opens the valve 20a for a predetermined amount of time to discharge the rinsing water retained in the first tank 21a and then closes all valves 20a to 20c.

The operation of the thus-configured endoscope system 1 according to this embodiment will be described below.

To acquire an image of an image-acquisition subject in a body cavity of a living body using the endoscope system 1 according to this embodiment, first, the insertion portion 2 is inserted into the body cavity, and the tip 2a is pointed toward the image-acquisition subject in the body cavity. In this state, the light source unit 4 and the control unit 6 are operated, and, by operating the light-source control circuit 11, the illumination light source 9 and the excitation light source 10 are operated to generate illumination light and excitation light.

For reflected-light examination carried out by emitting illumination light, after rinsing is carried out while confirming the rinsing position using the reflected light, two types of fluorescent probe solutions are sprayed. Then, after spraying the two types of fluorescent probes, examination is changed to fluorescence examination and the spraying condition of the fluorescent probes is confirmed using fluorescence before carrying out rinsing of the sprayed area. Subsequently, fluorescence examination of the sprayed area is carried out after the sprayed area is rinsed.

The illumination light and excitation light generated at the light source unit 4 are conveyed to the tip 2a of the insertion portion 2 via the light guide 8 and are emitted to the image-acquisition subject from the tip 2a of the insertion portion 2.

When the image-acquisition subject is irradiated with excitation light, the two types of fluorescent probes permeating the image-acquisition subject are simultaneously excited, and two types of fluorescence are simultaneously generated at the image-acquisition subject, as shown in FIG. 2. The two types of fluorescence generated at the image-acquisition subject are collected by the image-acquisition optical system 12 of the image-acquisition unit 3, transmitted through the excitation-light cut filter 13, and then split into two different wavelength bands by the dichroic prism 30. The fluorescence in a wavelength band of 400 nm or more to 720 nm or less is mainly captured by the image-acquisition device 14a, and the fluorescence in a wavelength band of 730 nm or more to 800 nm or less is mainly captured by the image-acquisition device 14b. In either case, the fluorescence is captured in a mixed state and is stored in the first frame memory 17a and the second frame memory 17b, respectively.

In such a case, part of the excitation light incident on the image-acquisition subject is reflected at the image-acquisition subject and enters the image-acquisition unit 3 together with the fluorescence. However, since the excitation-light cut filter 13 is provided in the image-acquisition unit 3, the excitation light is blocked and is prevented from entering the image-acquisition devices 14a and 14b.

At this point, the image processing circuit 18 receives fluorescence image information from the first and second frame memories 17a and 17b, and carries out calculation based on Equation 1, to calculate the concentrations N1 and N2 of the AlexaFluor680-based fluorescent probe and the AlexaFluor700-based fluorescent probe.

With the endoscope system 1 according to this embodiment, individual concentration information for each fluorescent probe can be calculated on the basis of the fluorescence image information acquired in a mixed state. Therefore, without using a device such as a variable spectroscopy device, the molecular distribution associated with cancer cells due to the fluorescent probes can be easily examined on the basis of fluorescence in wavelength bands that are close to or overlap each other such that they cannot be split even by fine control of the variable spectroscopy device.

The concentration information N1 and N2 calculated by the image processing circuit 18 is output to the first and second channels in the display unit 7 and are displayed on the display unit 7.

In this way, individual images showing the molecular distribution associated with cancer cells due to each fluorescent probe are displayed on the display unit 7 in an overlapping manner.

As a result, when fluorescence due to two fluorescent probes is generated in the same area, it can be easily confirmed that there is a high probability that cancer cells exist in that area. On the other hand, in an area where fluorescence due to only one fluorescent probe is generated, it can be determined that the probability of cancer cells existing in the area is low. Therefore, according to the present invention, there is an advantage in that the diagnostic performance can be improved by simultaneously using two types of fluorescent probes.

When the image-acquisition subject is irradiated with illumination light, the illumination light is reflected at the surface of the image-acquisition subject, collected at the image-acquisition optical system 12, and transmitted through the excitation-light cut filter 13. Then, the reflected light transmitted through the excitation-light cut filter 13 and the dichroic prism 30 enters the image-acquisition device 14a. In this way, reflected-light image information is acquired. In the wavelength band used for illumination light at this time, the transmittance of the dichroic prism 30 is 80% or more and reflectance is 1% or less; therefore, most of the reflected-light image information is received by the image-acquisition device 14a and almost none enters the image-acquisition device 14b. Therefore, a reflected-light image can be acquired based on only the image information of the image-acquisition device 14a.

The acquired reflected-light image information is stored in the third frame memory 17c, is output on the third channel of the display unit 7 by the image processing circuit 18, and is displayed on the display unit 7.

In this way, together with the image showing the molecular distribution associated with cancer cells due to the fluorescent probes, the actual external image of the examined target obtained with illumination light can be displayed in an overlapping manner, and the area where there is a high probability of cancer cells existing can be examined in relation to the actual external image.

In the endoscope system 1 according to this embodiment, as described above, reflected-light examination is carried out before fluorescence examination by operating the light-source control circuit 11 and the valve control circuit 16. In reflected-light examination, the light-source control circuit 11 operates the illumination light source 9 to irradiate the examined target with illumination light.

Then, when switching from reflected-light examination to fluorescence examination, before emitting excitation light, the valve control circuit 16 opens the valve 20a, while the illumination light source 9 emits illumination light, in order to discharge rinsing water retained in the first tank 21a from the tip 22a of the fluid supplying tube 22 to the examined target to rinse the surface of the examined target.

In this case, according to this embodiment, since the examined target is rinsed while the illumination light source 9 emits illumination light, the affected area can be easily confirmed, and the area to be sprayed with fluorescent probe solution can be rinsed while observing it.

The fluorescent probe solutions are also sprayed while the illumination light source 9 emits illumination light. Therefore, small amounts of fluorescent probe solution can be accurately sprayed at the required areas, without missing the position of the examined target, by opening the second and third valves 20b and 20c while confirming the position of the rinsed examined target. In this way, waste of expensive fluorescent probes can be prevented.

Subsequently, when the examined target is irradiated with excitation light by operating the excitation light source 10 with the light-source control circuit 11, the valve control circuit 16 receives a signal from the light-source control circuit 11 and turns off the valves 20a to 20c.

In such a case, according to this embodiment, after the fluorescent probe solutions are sprayed, the excitation light source 10 emits excitation light before rinsing; therefore even when the fluorescent probes are transparent, the spraying condition can be confirmed by fluorescence.

In the endoscope system 1 according to this embodiment, since the wavelength band of excitation light is on the longer wavelength side than the near-infrared band, the autofluorescent materials that originally exist in the examined target are not excited, and thus there is an advantage in that an even clearer image can be acquired by preventing the generation of autofluorescence.

In this embodiment, since two types of fluorescent probes are excited by one type of excitation light, it is not necessary to provide excitation-light sources of two different wavelengths.

In this embodiment, since the dichroic prism 30 has a characteristic of transmitting almost the entire visible band, the image-acquisition device 14a can be also used for normal optical examination in the visible band. Therefore, another image-acquisition device for normal optical examination does not have to be provided in addition to the image-acquisition device 14a for fluorescence examination.

In the endoscope system 1 according to this embodiment, the examined target is irradiated with one type of excitation light and illumination light, and an image showing the concentration distribution of two types of fluorescent probes and a reflected-light image are displayed in an overlapping manner. Instead, however, a third fluorescent probe may be used instead of illumination light, and second excitation light that excites the third fluorescent probe may be emitted. At this time, by using a fluorescent probe that generates fluorescence in a wavelength band different from those in which the first and second fluorescent probes generate fluorescence, as the third fluorescent probe, the spectral overlap between the fluorescent agents does not occur, and thus examination using three types of fluorescent probe with even better diagnostic performance can be carried out.

In this embodiment, the examined target is irradiated with excitation light and illumination light, and an image showing the concentration distribution of fluorescent probes and a reflected-light image are displayed in an overlapping manner. Instead, however, second excitation light that generates autofluorescence at the examined target may be emitted.

Since autofluorescence has a wavelength band far away from the agent fluorescence, which is located in the near-infrared band, it can be detected without causing the spectral overlap between the fluorescent agents with the agent fluorescence.

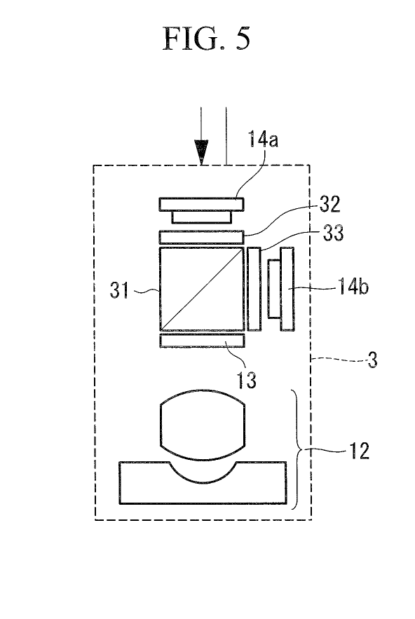

As shown in FIG. 5, instead of the dichroic prism 30, a beam splitter 31 may be used, a first filter 32 may be provided immediately before the image-acquisition device 14a, and a second filter 33 may be provided immediately before the image-acquisition device 14b.

At this time, the beam splitter 31 has a characteristic of splitting light from the examined target substantially equally into transmitted light and reflected light.

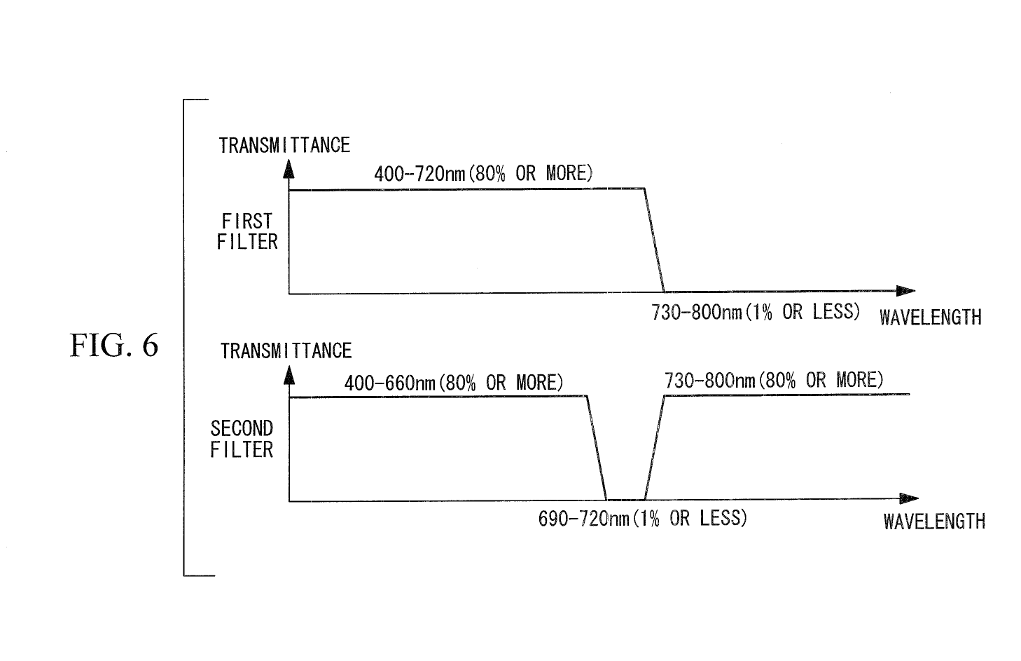

As shown in FIG. 6, the first filter 32 has a characteristic with a transmittance of 80% or more in a wavelength band of 400 nm or more to 720 nm or less and a transmittance of 1% or less in a wavelength band of 730 nm or more to 800 nm or less.

The second filter 33 has a characteristic with a transmittance of 80% or more in a wavelength band of 400 nm or more to 660 nm or less, a transmittance of 1% or less in a wavelength band of 690 nm or more to 720 nm or less, and a transmittance of 80% or more in a wavelength band of 730 nm or more to 800 nm or less.

In this way, fluorescence examination equivalent to that in the above-described embodiment can be carried out. Normally, when a light beam is split at the beam splitter 31, the light intensity is substantially halved. However, when carrying out normal optical examination in the visible band, examination is possible with sufficient light intensity even after the light is split at the beam splitter 31 by adding the images acquired by the image-acquisition device 14a and the image-acquisition device 14b and displaying them.

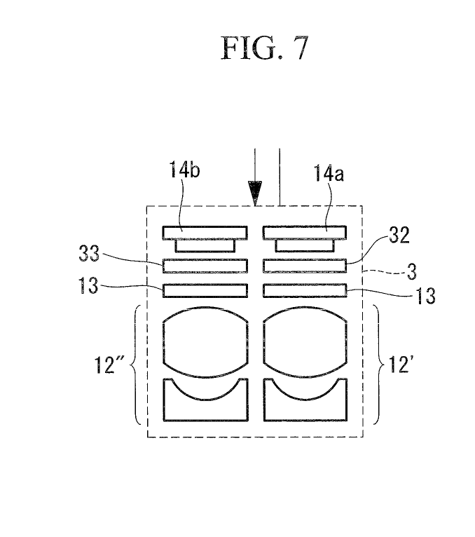

Moreover, as shown in FIG. 7, first and second image-acquisition optical systems 12′ and 12″ may be provided. The light from the examined target collected by the first image-acquisition optical system 12′ is transmitted through the excitation-light cut filter 13 and the first filter 32 and is received by the image-acquisition device 14a. Similarly, the light from the examined target collected by the second image-acquisition optical system 12″ is transmitted through the excitation-light cut filter 13 and the second filter 33 and is received by the image-acquisition device 14b.

The excitation-light cut filter 13 has a transmittance characteristic with a transmittance of 80% or more in a wavelength band of 400 nm or more to 670 nm or less, an OD value of 4 or more (=transmittance of 1×10⁻⁴ or less) in a wavelength band of 680 nm or more to 700 nm or less, and a transmittance of 80% or more in a wavelength band of 710 nm or more to 800 nm or less.

The first filter 32 has a characteristic with a transmittance of 80% or more in a wavelength band of 400 nm or more to 720 nm or less, and a transmittance of 1% or less in a wavelength band of 730 nm or more to 800 nm or less.

The second filter 33 has a characteristic with a transmittance of 80% or more in a wavelength band of 400 nm or more to 660 nm or less, a transmittance of 1% or less in a wavelength band of 690 nm or more to 720 nm or less, and a transmittance of 80% or more in a wavelength band of 730 nm or more to 800 nm or less.

With this configuration, fluorescence examination and normal examination equivalent to those in the above-described embodiment can be carried out.

1. An endoscope system configured to insert at least a part thereof into a body cavity of a living body and to acquire an image of an image-acquisition subject in the body cavity, the endoscope system comprising:
a light source unit configured to emit excitation light for exciting two or more different types of fluorescent agents having different optical characteristics;
two or more image-acquisition units provided at a section inserted in the body cavity and configured to simultaneously capture fluorescence emitted from the image-acquisition subject as fluorescence in two or more different wavelength bands;
a storage unit configured to store information associated with the relative relationship between the intensity of fluorescence generated when excited by the excitation light and the concentrations of the fluorescent agents; and
a concentration-information calculating unit configured to calculate and output concentration information of the fluorescent agents on the basis of fluorescence intensity of images in two or more wavelength bands captured by the image-acquisition units and the information associated with the relative relationship stored in the storage unit.

2. The endoscope system according to claim 1, wherein the information associated with the relative relationship is information about the ratio of the intensity of the fluorescence generated when excited by the excitation light and the concentration of the fluorescent agents.

3. The endoscope system according to claim 1, further comprising:
a display configured to display the concentration information calculated and output by the concentration-information calculating unit.

4. The endoscope system according to claim 3, wherein
the display has a plurality of channels corresponding to display colors, and
the concentration information corresponding to the fluorescent agents are assigned to and output on the channels.

5. The endoscope system according to claim 1, wherein the wavelength of the excitation light is set longer than the near-infrared band.

6. The endoscope system according to claim 1, wherein the information associated with the relative relationship is stored for each of two or more wavelength bands received by each of the two or more image-acquisition units, respectively.

7. An endoscope system configured to insert at least a part thereof into a body cavity of a living body and to acquire an image of an image-acquisition subject in the body cavity, the endoscope system comprising:
a light source unit configured to emit excitation light for exciting two or more different types of fluorescent agents having different optical characteristics;
two or more image-acquisition units provided at a section inserted in the body cavity and configured to simultaneously capture fluorescence emitted from the image-acquisition subject as fluorescence in two or more different wavelength bands;
a storage unit configured to store information associated with the relative relationship between the intensity of fluorescence generated when excited by the excitation light and each of the fluorescence in two or more different wavelength bands; and
a concentration-information calculating unit configured to calculate and output concentration information of the fluorescent agents on the basis of fluorescence intensity of images in two or more wavelength bands captured by the image-acquisition units and the information associated with the relative relationship stored in the storage unit.
